(19) United States
(12) Patent Application Publication
(10) Pub. No.: US 2025/0172494 A1
(43) Pub. Date: May 29, 2025

(54) METHOD FOR DETERMINING A SCATTERED LIGHT PARAMETER AND MEASURING ARRANGEMENT FOR PERFORMING THE METHOD

(71) Applicant: Endress+Hauser Conducta GmbH+Co. KG, Gerlingen (DE)

(72) Inventors: Matthias Großmann, Vaihingen-Enz (DE); Thilo Krätschmer, Gerlingen (DE); Andreas Bayer, Gerlingen (DE); Alexander Opolka, Karlsruhe (DE)

(21) Appl. No.: 18/961,208

(22) Filed: Nov. 26, 2024

(30) Foreign Application Priority Data
Nov. 27, 2023 (DE) 10 2023 133 015.2

(51) Int. Cl. G01N 21/47; G01N 21/49; G01N 33/18

(52) U.S. Cl. CPC G01N 21/4738; G01N 21/49; G01N 33/18

(57) ABSTRACT

A method for determining a scattered light parameter, for example the turbidity, in a medium using a measuring arrangement, for example a turbidity sensor, the method including the steps of: transmitting excitation light into the medium, wherein the excitation light is scattered in the medium; receiving the light scattered in the medium, resulting in an optical path of excitation light and scattered light; generating interference in the optical path; receiving the light now scattered in the medium; and determining the scattered light parameter, for example the turbidity, based on the scattered light, accounting for the influence of the interference. The present disclosure further discloses a measuring arrangement, for example a turbidity sensor, for performing the method.

### CROSS-REFERENCE TO RELATED APPLICATION

The present application is related to and claims the priority benefit of German Patent Application No. 10 2023 133 015.2, filed on Nov. 27, 2023, the entire contents of which are incorporated herein by reference.

### TECHNICAL FIELD

The present disclosure relates to a method for determining a scattered light parameter, for example turbidity, and to a measuring arrangement for performing the method.

### BACKGROUND

In the following, the problem to be solved is described using a turbidity measurement as an example. The ISO 7027 standard, which is relevant for turbidity measurements in liquids, prescribes scattered light measurement at an angle of 90° for the measurement of low turbidity. Optical sensors for measuring the turbidity of a liquid which meet this standard have the disadvantage that the signal curve is not clear in all media: As the turbidity of the liquid increases, the scattered light signal initially increases up to a maximum value. However, once this limit is exceeded, the signal decreases again despite increasing turbidity, as less and less scattered light reaches the sensor's detector due to multiple scattering.

Due to the ambiguous signals, the measurement cannot distinguish between very low and very high turbidity values and thus the turbidity value and/or the solids content of a liquid cannot be clearly determined. For example, black medium that is heavily contaminated with particles is incorrectly interpreted as clean, clear liquid.

Since international regulations and legal requirements often require turbidity measurement according to ISO 7027, and thus only a single scattered light signal with a 90° angle can be used, additional measuring devices usually must be installed to obtain further information about the actual turbidity value or solids content of the medium through additional measured variables. This entails considerable additional costs and installation effort for the operators.

### SUMMARY

The present disclosure is based on the object of performing optical scattered light measurements with only one measuring channel (for example at 90° according to ISO7027) in such a way that a clear distinction can be made between a medium with low turbidity (e.g., clear water) and a liquid with very high turbidity (e.g., black wastewater with a high particle content) and/or the turbidity value/the solids content of the liquid can be detected.

The object is achieved by a method comprising the steps of:
transmitting excitation light into the medium, such that the excitation light is scattered in the medium; receiving the light scattered in the medium, resulting in an optical path of excitation light and scattered light; generating interference in the optical path; receiving the light now scattered in the medium; and determining the scattered light parameter, for example the turbidity, based on the scattered light, taking into account the influence of the interference.

In the disclosed method, the unambiguous distinction between a medium with low turbidity (e.g., clear water) and a liquid with very high turbidity or absorption (e.g., contaminated, black wastewater with a high particle content) is achieved by generating defined interferences in the optical path of the sensor and evaluating the reactions to these interferences.

These interferences are reflections, scattering or other optical interactions with fixed or moving components, walls, other mechanical elements or air bubbles or induced anomalies such as turbulence. In at least one embodiment, the interference can be achieved by a targeted manipulation of the turbidity value (e.g., addition of ultrapure water).

Although the measurement signals in the very different media can be the same without interference due to the problem described above, optical interference results in significantly different signals, the changes of which have different signs, which allow a clear detection of the degree of contamination of the liquid: For example, while the interference caused by additional wall effects in media with low turbidity leads to an increase in the measurement signal, the measurement signal decreases in highly turbid media.

The following describes the embodiments.

At least one embodiment provides that the method further comprises the step of moving an interference element, for example an element that reflects and/or scatters the excitation light, into the optical path, such that the interference is generated.

At least one embodiment provides that the method further comprises the step of moving the measuring arrangement in the direction of an interference element, for example in the direction of an element that reflects and/or scatters the excitation light, so that excitation light is reflected and/or scattered by this element, thereby generating the interference.

At least one embodiment provides that the interference is designed as electro-optical interference, and that interference light is received in addition to the scattered light.

At least one embodiment provides that the interference is generated temporarily and regularly.

At least one embodiment provides that the method further comprises the step of introducing ultrapure water or a secondary medium with a defined turbidity into the medium.

The object is further achieved by a measuring arrangement, for example a turbidity sensor, for performing the method as described above for determining a scattered light parameter, for example the turbidity, in a medium, the measuring arrangement comprising at least one light source which transmits excitation light into the medium and in the direction of the medium surface; at least one photodiode which receives light scattered in the medium and converts it into an electrical signal, thereby establishing an optical path proceeding from the light source, through the medium, to the photodiode; an interference unit which is designed to generate interference in the optical path; and a data processing unit which determines the scattered light parameter, for example the turbidity, from the electrical signal on the basis of the scattered light, taking into account the influence of the interference.

At least one embodiment provides that the light source transmits the excitation light into the medium via an optical window, wherein the photodiode receives scattered light from the medium via the same window or a separate optical window, wherein the interference unit is designed as a reflective and/or scattering interference element, for example as a wiper which cleans the optical window, wherein the interference element generates the interference when it is positioned in front of the optical window, and thus in the optical path.

At least one embodiment provides that the interference unit is arranged outside of the medium.

At least one embodiment provides that the light source transmits the excitation light into the medium via an optical window, wherein the photodiode receives light scattered by the medium via the same window or a separate optical window, wherein the interference unit is designed as an air cleaning unit and blows air in the direction of the optical window, wherein the air cleaning unit generates the interference when there is air in front of the optical window, and thus in front of the optical path.

At least one embodiment provides that the interference unit is designed as a movement element and moves the measuring arrangement in the direction of interference element, for example in the direction of an element that reflects and/or scatters the excitation light, so that excitation light is reflected and/or scattered by this element, such that the interference is generated.

At least one embodiment provides that the interference unit is designed as an interference light source and transmits interference light in the direction of the photodiode, such that the interference is generated.

At least one embodiment provides that the interference unit is designed as a scattering or reflection unit and the measuring arrangement with the light source is positioned relative to the scattering or reflection unit in such a way that a long-term measured value offset results at least for a medium that is slightly or not at all turbid.

One embodiment provides that the interference unit is designed as a secondary medium dispensing unit, which introduces ultrapure water or a secondary medium with a defined turbidity into the medium and generates the interference.

### BRIEF DESCRIPTION OF THE DRAWINGS

The present disclosure is explained in more detail with reference to the following figures.

FIG. 1 shows a general view of an arrangement for measuring scattered light, having an optical path.

FIGS. 2a and 2b show the measured scattered light intensity at different turbidities according to the prior art (2a) and by means of the measuring arrangement and method according to the present disclosure (2b).

FIG. 3 shows an embodiment of the measuring arrangement according to the present disclosure.

FIGS. 4a and 4b show an embodiment of the measuring arrangement according to the present disclosure with the effect in less turbid medium (4a) and more turbid medium (4b).

FIGS. 5a and 5b show an embodiment of the measuring arrangement (5a) and the effect (5b, symbolically).

FIGS. 6a and 6b show an embodiment of the measuring arrangement with the effect in less turbid medium (6a) and more turbid medium (6b).

FIGS. 7a and 7b show an embodiment of the measuring arrangement with the effect in less turbid medium (7a) and more turbid medium (7b).

FIGS. 8a and 8b show an embodiment of the measuring arrangement with the effect in less turbid medium (8a) and more turbid medium (8b).

FIGS. 9a and 9d show an embodiment of the measuring arrangement with the effect in less turbid medium (9a, 9b) and more turbid medium (9c, 9d).

FIG. 10 shows the measured scattered light intensity at different turbidities for the measuring arrangement of FIG. 9.

In the figures, the same features are labeled with the same reference signs.

### DETAILED DESCRIPTION

Any light striking particles suspended in a liquid is scattered. The intensity of this light scattering is used in optical turbidity measurement as a direct measure for the determination of turbidity. Different measurement angles are used for different applications—in part due to national legal provisions. For example, the 90° scattered light is used in drinking water applications, among others. Breweries often use a scattered light angle in the range of 11° to 25°. In measurements in sludges, a backscattering angle of >90° (e.g., 135°) is mostly used. “FNU” units (Formazin Nephelometric Units) are often used as reference measurement or for turbidity values.

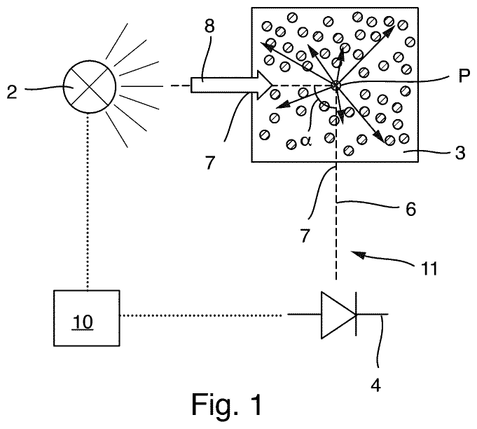

Typically, a turbidity sensor based upon scattered light measurement can be symbolically illustrated as in FIG. 1. From the light source 2, excitation light 8 (see thick arrow) is radiated through a window 7 that is transparent to the excitation light 8 into a measurement chamber. There, the light is scattered by particles in the medium 3 being measured at a scattering point P and at a measuring angle α (here 90° as an example) and converted into “scattered light 6” (dashed vertical line). In reality, the scattering is not a single line (ray of light) as shown, but a diffuse volume.

The scattered light 6 passes through a window 7 that is transparent to it (it is possible to use only a single window), for example via an aperture(s) or lens(es), to a receiver 4. The light intensity arriving at the receiver 4 is a measure of the turbidity. The light path from the light source 2, through a window 7 into the medium 3, to the scattering point P, through the medium 3 and through a window 7 to the receiver 4 is generally referred to as the “optical path 11” (dashed line). As mentioned above, scattering in reality does not result in just a single line (ray of light), but rather a diffuse volume. The “optical path 11” therefore encompasses the entirety of this diffuse volume. As explained below, interference is generated in this volume (i.e., in the optical path) in such a way that a receiver receives the scattered light caused by the interference.

The measuring arrangement 1 according to the present disclosure is also referred to as a sensor or turbidity sensor below. The turbidity sensor 1 is arranged on a container, for example on a pipe or in a tub or open channel. This is carried out via fastening means, such as a flange. The turbidity sensor can also be arranged on an immersion or quick-change fitting or the like. The medium 3 being measured is located in the container; for example, the medium 3 may flow through the container. The medium 3 to be measured is mostly a liquid—often process and wastewater. The arrangement is, however, also used in fresh water, for example, drinking water. When used on a container, the turbidity sensor is arranged substantially perpendicular to the longitudinal axis of the container, such as the pipe. The turbidity sensor comprises a housing 5. Stainless steel, plastic, or a ceramic can be used as materials for the arrangement 1, for example for the housing 5. The materials are selected such that they are suitable for use in the given application.

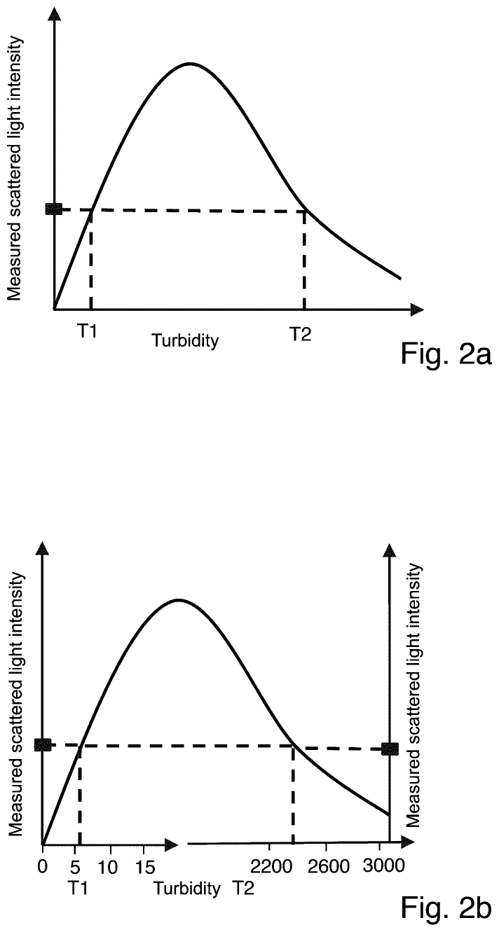

The arrangement 1 comprises at least one light source 2 and a receiver 4. Via electrical connections, the light source and the receiver are connected to a data processing unit 10 (not shown). The data processing unit 10 determines the measured value to be determined, e.g. the turbidity, from the electrical signal of the scattered light. This measurement is determined using a calibration model, which combines the measured information regarding the received scattered light intensities and uses this to determine the turbidity. FIGS. 2a and 2b, for example, show the relationship between measured scattered light intensities and the corresponding turbidity. In FIG. 1, the electrical connection of the data processing unit 10 with the light source 2 and the receiver 4 is shown in dotted lines.

The receiver 4 is, for example, designed as a photodiode, which generates a receiver signal, such as a photocurrent or a photovoltage, from the light received (generally: an electrical signal).

The light source 2, often an LED, transmits light toward the medium 3. In this respect, “light,” within the context of this application, should not be limited to the visible range of the electromagnetic spectrum, but is to be understood as electromagnetic radiation of any wavelength, for example in the ultraviolet (UV) and in the infrared (IR) wavelength range. For example, a wavelength of the light may be 860 nm. The arrangement may also include other optical components in the beam path after the light source, such as filters or one or more lenses. Corresponding components are also arranged on the receiver side, on the photodiode 4.

In FIG. 2a, the measured scattered light intensity is the same for two different turbidity values, so it is not possible to distinguish between turbid and clear water purely using measurement (prior art).

In the measuring arrangement 1 and the method of the present disclosure, the clear distinction between a medium with low turbidity (e.g., clear water) and a liquid with very high turbidity or absorption (e.g., contaminated, black wastewater with a high particle content) is achieved by generating defined interferences in the optical path 11 of the sensor 1 and evaluating the reactions to these interferences.

Since the disclosed solution eliminates the ambiguity of the signal curve, the measuring range of the sensor 1 can be significantly increased, because even high turbidities on the decreasing part of the signal curve can be measured. While with state-of-the-art turbidity sensors only the measured values of one side of the curve can be used, in this case both sides of the curve can be used due to the clear assignment of the measured scattered light intensity to one of the two sides of the signal curve. This results in a significantly larger measuring range on the one hand. On the other hand, the assignment of a measured scattered light intensity to an incorrect turbidity value is avoided. FIG. 2b shows that both the left and right half of the scattered light intensity/turbidity curve can be used.

In addition, the discreteness of the signal curve simplifies model selection for the user. A model which is, for example, optimized for the measurement of clear media (e.g., drinking water) can be excluded in advance or actively blocked by the software of sensor 1 if the sensor detects a heavily contaminated medium.

The above-mentioned interference of the optical path 11 can be generated by the interference unit 15. This can be done temporarily, for example.

At least one embodiment comprises an interference element 12 which reflects and/or scatters and is designed, for example, as a movable, external element in the optical path 11 of a fixedly positioned sensor 1. As mentioned above, the optical path 11 comprises the entirety of the diffuse volume of the scattered light, including the interference generated, such that the receiver receives the scattered light caused by the interference.

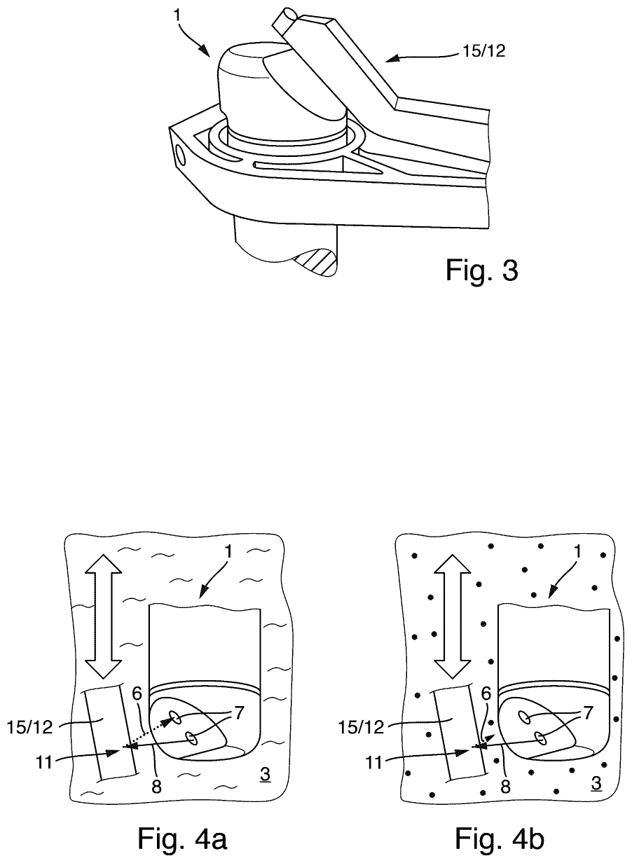

FIG. 3 shows one embodiment. For example, a wiper cleaning unit, or its wiper arm and wiper lip, installed on the sensor 1 can be guided in a defined manner over the optical windows 7 and the measurement signal and/or the reaction to this optical interference can be evaluated. The wiper serves as the interference unit 15 and/or as the interference element 12, which reflects and/or scatters. In this way, the cleaning of the optical windows 7 can be profitably combined with the detection of the degree of contamination of the medium 3.

Furthermore, any other movable elements (plates, wings, etc.) can be temporarily pivoted into the optical path of the sensor. The movement of an interference element 12 into the optical path 11 is symbolized by the vertical arrows in FIGS. 4a and 4b. FIG. 4a shows the effect in less turbid medium—FIG. 4b in more turbid medium. The effects are symbolized by the arrows of different thicknesses of the excitation light 8 and the reflected light 6. In a very turbid medium (FIG. 4b) the light is absorbed more strongly and less or no light reaches the receiver 4.

In all figures, a thick arrow means “more” light, a thin arrow means “less light”. In some figures, the arrow, which represents light, breaks off. This is, of course, only to be understood symbolically. Physically, the light attenuates along the arrows (or spherically).

When using a fitting made of transparent or partially transparent materials, the movable elements 12 described above can also be mounted outside the medium 3 to generate interference. In this embodiment, the sensor 1 must be positioned such that it detects the interference element 12 through a wall in media 3 with low turbidity (e.g., clear water) and/or reacts to the interference in its optical path 11 by an increase in the measuring signal.

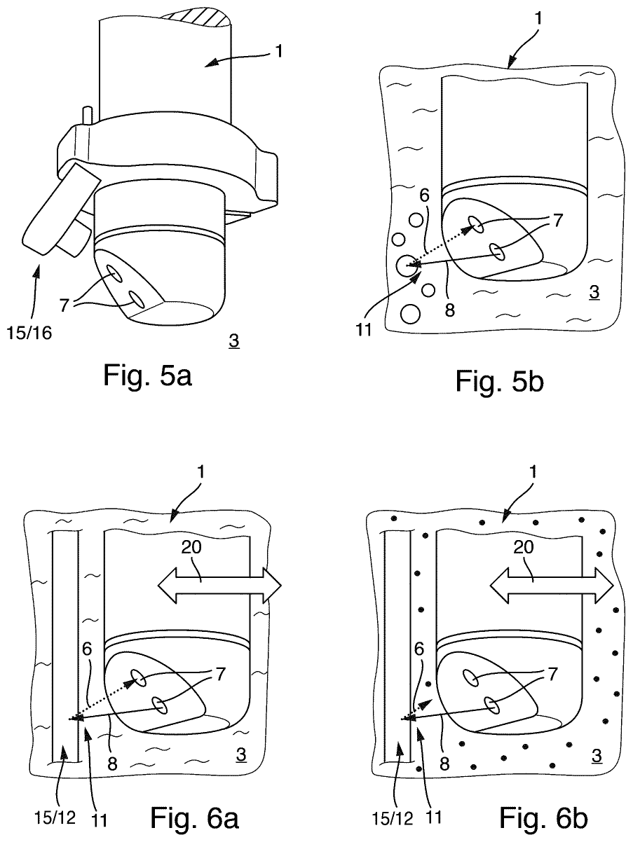

An embodiment is shown in FIGS. 5a and 5b with an air cleaning unit 16 attached to the sensor head, which functions as the interference unit 15. FIG. 5a shows this design; FIG. 5b shows this design symbolically with air bubbles. The air cleaning unit 16 temporarily blows air bubbles into the optical path 11 of the sensor 11. The subsequent measurement signals with and without interferences are evaluated.

In one embodiment, the sensor 1 as a movable element is temporarily moved to a fixedly positioned interference unit 15, which is reflective (reference number 12). For example, the sensor 1 can be moved or rotated via a movable holding device 20 in such a way that the wall of a vessel or a tub temporarily disturbs the signals in the optical path 11 in order to evaluate the reaction to this interference. FIG. 6a shows the effect in less turbid medium—FIG. 6b in more turbid medium. The movement of the sensor 1 into the optical path 11 by means of a movement element 20 is symbolized by the horizontal arrows in FIGS. 6a and 6b.

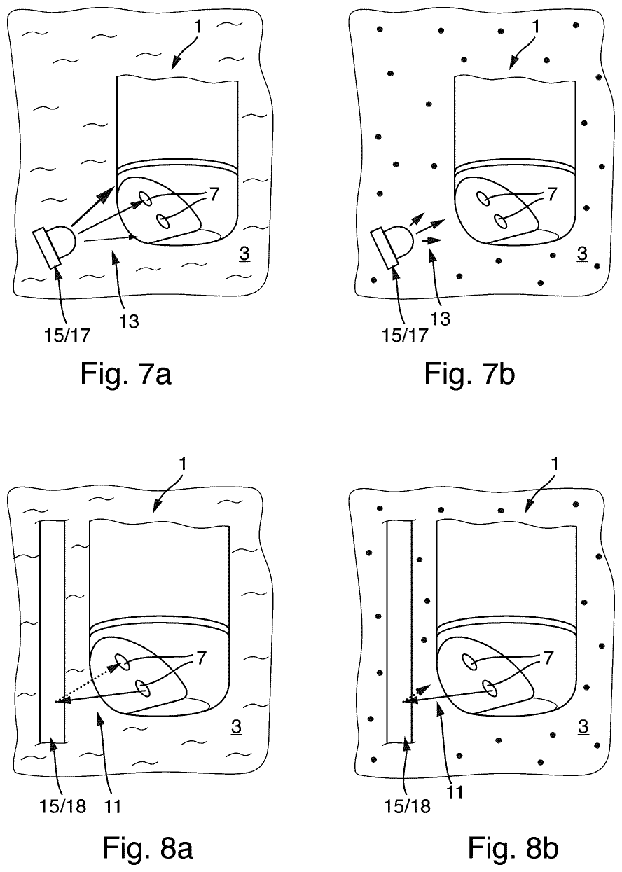

FIGS. 7a and 7b shows one embodiment. This temporarily generates an electro-optical interference, which causes different reactions in a fixedly positioned sensor 1 depending on the medium 3. FIG. 7a shows the effect in a less turbid medium 3—FIG. 7b in a more turbid medium 3. For example, a light source 17 (LED, etc.) can be temporarily switched on as the interference element 15, which leads to a significant increase in the measured value in a clear medium, while the measured value changes hardly or only slightly in a heavily contaminated medium.

As an embodiment of the above-described interferences temporarily introduced into the optical path 11 with different states (with or without interference), the interference can also be introduced permanently. This has the advantage that no additional movement elements, controls, etc. are required.

For example, a sensor can be positioned close enough to a vessel, tub, or fitting wall 18 that in clear water a defined offset of, for example, +1 FNU results from the scattering or reflection on the pool wall. The wall 18 then serves as the interference element 15 (“scattering or reflection unit”). This is shown in FIGS. 8a and 8b. FIG. 8a shows the effect in less turbid medium 3—FIG. 8b in more turbid medium 3.

A heavily contaminated medium can then be clearly detected by the measured value falling below this minimum value, since reflections on the tank wall do not penetrate through to the sensor's detector. Of course, the set offset can be corrected in the signal processing chain so that the correct turbidity value is output in the final measured value display.

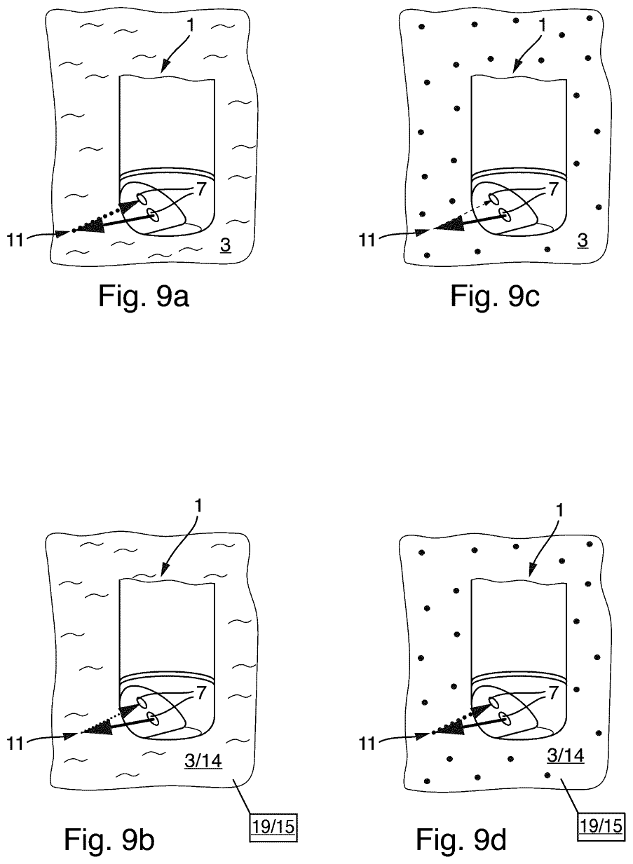

In at least one embodiment, the interference is generated in the form of a short-term, active manipulation of the turbidity value (of a liquid with a defined turbidity; reference numeral 14). A secondary medium dispensing unit 19 as the interference unit 15 adds the medium 14. This is shown in FIGS. 9a-9d. FIGS. 9a and 9b show the effect in less turbid medium 3—FIGS. 9c and 9d in more turbid medium 3. FIGS. 9a and 9c show the situation without interference; FIGS. 9b and 9d with the interference by addition of a second medium with defined turbidity.

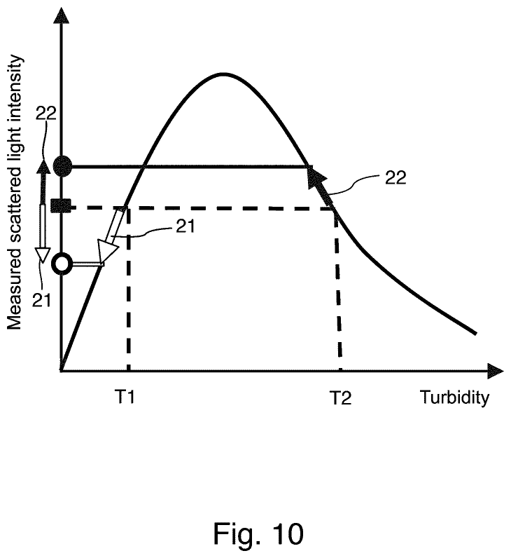

FIG. 10 shows the measured scattered light intensity compared to the turbidities T1 and T2 and the effect of the introduction of ultrapure water 14 according to the embodiments in FIG. 9a-9d. For example, the addition of ultrapure water in a medium with low turbidity (FIGS. 9a and 9b) leads to a decrease in the measured value (reference number 21), while the measured value in a heavily contaminated medium (FIGS. 9c and 9d) increases (reference number 22).

1. A method for determining a scattered light parameter in a medium using a measuring arrangement, the method comprising:
transmitting excitation light into the medium, wherein the excitation light is scattered in the medium;
receiving the light scattered in the medium, which defines an optical path of excitation light and scattered light;
generating interference in the optical path;
receiving the light now scattered in the medium; and
determining the scattered light parameter based on the light scattered in the medium, including influence of the interference.

2. The method according to claim 1, wherein the interference is a reflection, a scattering, or another optical interaction.

3. The method according to claim 1, further comprising moving an interference element into the optical path such that the interference is generated.

4. The method according to claim 3, further comprising moving the measuring arrangement in the direction of the interference element such that the excitation light is reflected and/or scattered thereon such that the interference is generated.

5. The method according to claim 1, wherein the interference is configured as electro-optical interference, and wherein interference light is received in addition to the scattered light.

6. The method according to claim 1, wherein the interference is generated temporarily and regularly.

7. The method according to claim 1, further comprising introducing ultrapure water or a secondary medium with a defined turbidity into the medium.

8. A measuring arrangement for performing the method according to claim 1, the measuring arrangement comprising:
at least one light source configured to transmit excitation light into the medium and in a direction of a surface of the medium;
at least one photodiode configured to receive the light scattered in the medium and to convert the light scattered in the medium into an electrical signal, thereby defining an optical path from the at least one light source, through the medium, and to the at least one photodiode;
an interference unit configured to generate interference in the optical path; and
a data processing unit configured to determine the scattered light parameter from the electrical signal based on the light scattered in the medium, taking into account the influence of the interference.

9. The measuring arrangement according to claim 8, wherein the at least one light source is configured to transmit the excitation light into the medium via a first optical window,
wherein the at least one photodiode receives the light scattered in the medium via the first optical window or a second optical window,
wherein the interference unit is a reflective and/or scattering interference element,
wherein the interference element generates the interference when positioned in front of the first optical window and in the optical path.

10. The measuring arrangement according to claim 9, wherein the interference unit is arranged outside the medium.

11. The measuring arrangement according to claim 8,
wherein the at least one light source is configured to transmit the excitation light into the medium via a first optical window,
wherein the at least one photodiode receives light scattered from the medium via the first optical window or a second optical window,
wherein the interference unit is an air cleaning unit configured to blow air in a direction of the first optical window or the second optical window,
wherein the air cleaning unit generates the interference when air is blown in front of the first optical window or second optical window and in the optical path.

12. The measuring arrangement according to claim 8 wherein the interference unit is a movement element configured to move the measuring arrangement in a direction of an interference element, which reflects and/or scatters the excitation light such that the excitation light is reflected and/or scattered, such that the interference is generated,
wherein the interference element generates the interference when positioned in front of the first optical window and in the optical path.

13. The measuring arrangement according to claim 8, wherein the interference unit is configured as an interference light source, which transmits interference light in a direction of the at least one photodiode such that the interference is generated.

14. The measuring arrangement according to claim 8, wherein the interference unit is a scattering or reflection unit, and the measuring arrangement is positioned relative to the scattering or reflection unit such that a permanent measured value offset results at least for a medium that is slightly or not at all turbid.

15. The measuring arrangement according to claim 8, wherein the interference unit is a secondary medium dispensing unit, which introduces ultrapure water or a secondary medium with a defined turbidity into the medium as to generate the interference.

16. The method according to claim 1, wherein the measuring arrangement includes a turbidity sensor and the scattered light parameter is turbidity.

17. The method according to claim 3, wherein the interference element is an element that reflects and/or scatters the excitation light into the optical path.

18. The method according to claim 3, wherein the interference element is moved in the direction of an element that reflects and/or scatters the excitation light.

19. The measuring arrangement according to claim 8, wherein the measurement arrangement includes a turbidity sensor and the scattered light parameter is turbidity.

20. The measuring arrangement according to claim 9, wherein the interference unit is embodied as a wiper, which is configured to clean the first optical window or second optical window.
